(19) United States
(12) Patent Application Publication
(10) Pub. No.: US 2005/0197606 A1
(43) Pub. Date: Sep. 8, 2005

(54) Adjustable soft neck brace

(76) Inventor: Alejandro Freire, Montebello, CA (US)

Correspondence Address: CHRISTIE, PARKER & HALE, LLP, PO BOX 7068, PASADENA, CA, 91109-7068, US

(21) Appl. No.: 10/795,896

(22) Filed: Mar. 8, 2004

(51) Int. Cl. A61F 5/00

(52) U.S. Cl. 602/18

(57) ABSTRACT

An adjustable neck brace system with a brace body, elongate inserts, and engaging members. The brace body is made of flexible material, has two side edged and a top and bottom edge, and has a plurality of tunnels formed therethrough, with each tunnel having a first, smaller internal diameter when in an unstretched condition. The inserts are received in the tunnels. The engaging members are affixable in the vicinity of the side edges of the brace body and are used for retaining the neck brace around a wearer's neck. By selecting inserts that have an outer diameter that is larger than the first, smaller internal diameter of the brace body in its unstretched condition, the tunnels will be stretched, which also stretches the brace body, thereby lengthening the distance between the bottom edge and top edge of the brace body.

### SUMMARY OF THE INVENTION

This invention relates to the field of neck and cervical braces, and more particularly to a neck and cervical brace that is made of a generally soft and pliable material and which can be adjusted in size and shape to provide for a wide range of patient needs, and which can be adjusted during a course of therapy.

There are numerous neck braces available for a wide variety of neck and cervical spine stabilizing purposes. Typically, these prior neck braces are manufactured from relatively stiff material to provide the necessary rigidity, and are padded with foam rubber material, gel material, and/or soft fabrics to provide for a more comfortable fit around a wearer's neck region.

These prior art neck braces tend to be relatively uncomfortable to a wearer since the padding on top of the relatively rigid and stiff material is not always comfortable to the patient. Furthermore, these prior neck braces have relatively little adjustability for a patient other than adjustability as to the diameter of the neck. For example, these prior art neck braces typically do not have adjustability as to their size and shape so that during the course of immobilization and other therapy, the shape and size of the neck brace can be adjusted to fit a patient's possibly changing needs. Moreover, in order to keep the costs of neck braces more affordable, most manufacturers offer a relatively small variety of sizes with little adjustability, thus, the fit is even less than perfect.

There accordingly remains a need for a soft neck brace which can be adjusted as to size and shape yet provides for a comfortable patient fit.

### BRIEF DESCRIPTION OF THE DRAWINGS

FIG. 1 is a front perspective view showing a patient wearing an exemplary adjustable soft neck brace of the invention.

FIG. 2 is a rear perspective view showing a patient wearing the exemplary adjustable soft neck brace of FIG. 2.

FIG. 3 is a right side view of the exemplary adjustable soft neck brace of FIG. 1

FIG. 4 is a detail of FIG. 3.

FIG. 5 is a cross sectional through view lines 5-5 of FIG. 4 showing smaller tubes inserted in tunnels therein.

FIG. 6 is an exemplary cross sectional view showing a tool used to insert tubing into a tunnel of an exemplary flexible neck brace of the invention.

FIG. 7 is a top plan view showing another exemplary adjustable soft neck brace of the invention.

FIG. 8 is a cross sectional through view lines 5-5 of FIG. 4 showing larger tubes inserted in tunnels therein.

### DESCRIPTION OF THE INVENTION

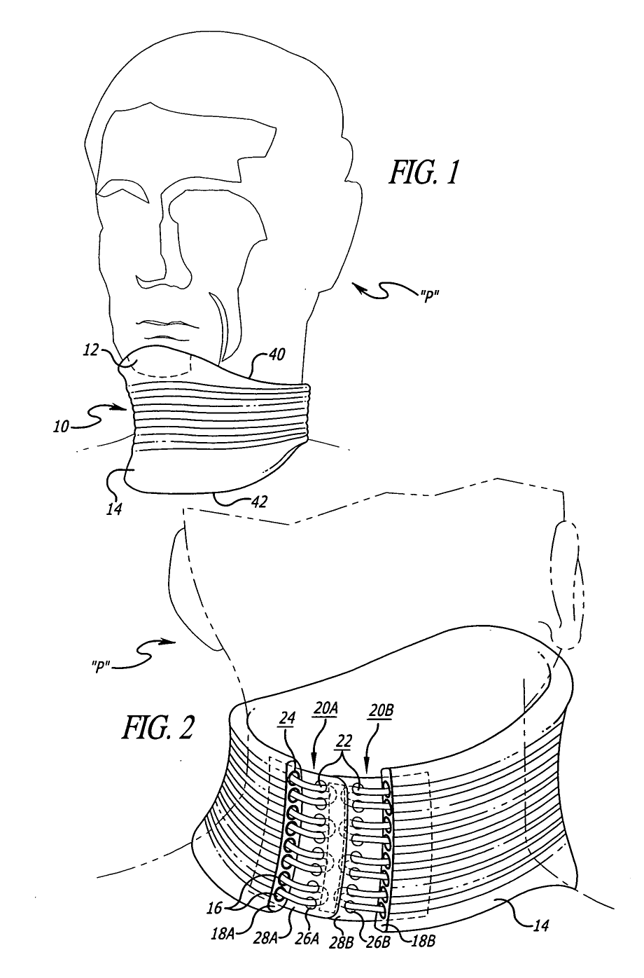

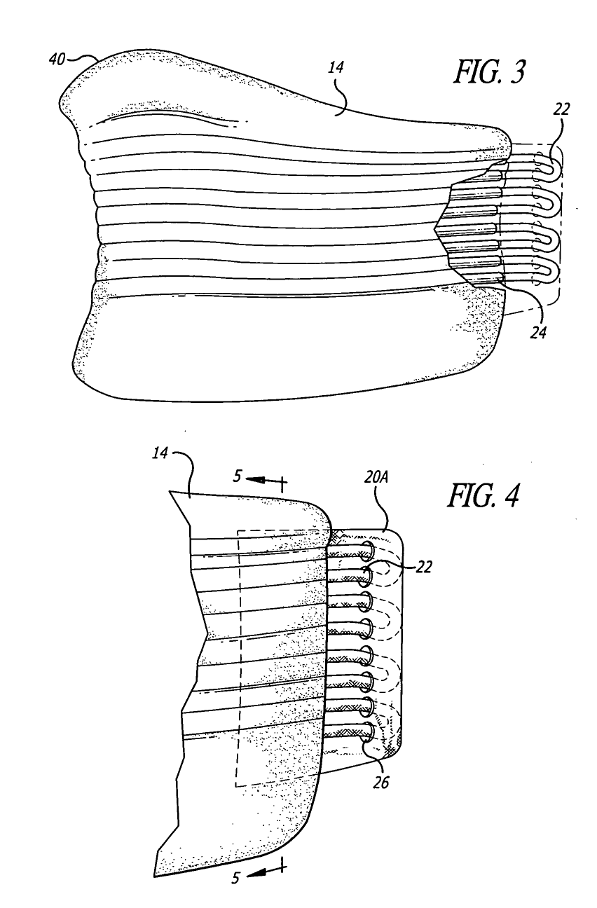

Turning to FIGS. 1 and 2, there are shown front and rear perspective views of a patient “P” wearing an exemplary adjustable soft neck brace 10 of the invention, with an optional chin cup 12. Adjustable soft neck brace 10 has a brace body comprising a soft and flexible portion 14 which extends around at least the front the patient's neck and at least partially around the side and back of the neck. Soft and flexible portion 14 can preferably extend from the vicinity of the patient's chin to the patient's lower neck or upper chest/shoulders in order to assist in stabilizing the patient's neck and/or head for a variety of injuries, conditions and treatments. Soft and flexible portion 14 can be made of a material such as silicon rubber or other soft and pliable plastics and rubbers. A series of spaced apart tunnels 16 are formed through soft and flexible portion 14 from its opposite ends 18A and 18B. Engaging members 20A and 20B are attachable to opposite ends 18A and 18B of soft and flexible portion 14. In the exemplary embodiment shown in FIGS. 1-5, a string or strings 22 are passed through tubing 24 inserted into tunnels 16, and string or strings 22 are looped through apertures 26A and 26B located in engaging members 20A and 20B, respectively. The use of a string or strings 22 passing through tubing 24 provides a way to adjust the fit and relative degree of tension of various horizontal regions of soft and flexible portion 14 relative engaging members 20A and 20B. For example, by tightening the portion of the strings in the central portion of the neck brace, that portion will be drawn in closer to engaging members 20A and 20B. Engaging members 20A and 20B can have detachable attachment means located thereon, such as hook and loop material, snaps, hooks, and/or other means and materials, so that engaging members 20A and 20B can be held together, and thereby hold soft and flexible portion 14 around patient's neck. As shown, engaging members 20A and 20B can comprise strips of material 28A and 28B, with apertures 26A and 26B and detachable attachment means. A portion of material 28A and 28B can underlie opposite ends 18A and 18B of soft and flexible portion 14. Soft and flexible portion 14 has an upper edge 40 and a lower edge 42. To fit the contour of a patient's neck and chest/shoulders, upper edge 40 and a lower edge 42 will preferably by curved, with a variety of vertical distances between the edges.

Turning to FIG. 5, there is shown a cross-sectional view of exemplary neck brace 10 along view lines 5-5 of FIG. 4. Neck brace is shown with a plurality of tubing 24 having a diameter t₁ inserted into tunnels 16. The soft and flexible 14 is stretchable, and its tunnels will be stretchable to receive different diameter tubing. The distance d₁ will be affected by the size of the diameter of the tubing inserted into tunnels 16. The tubes are shown with strings 22 passing through opening 44 of tubing 24.

Turning to FIG. 8, there is shown another cross-sectional view of exemplary neck brace 10 along view lines 5-5 of FIG. 4. Neck brace is shown with a plurality of larger diameter tubing 46 having a diameter t₂ inserted into tunnels 16. Since the soft and flexible neck brace 14 is stretchable, and its tunnels will be stretchable to receive this larger diameter tubing 46, and in the process, the distance d₂ will be stretched to be larger than distance d₁ (with smaller diameter tubing 24.) Tubing is shown with strings 22 passing through opening 48 therein. In addition to changing the distances d₁ and d₂, using different sized tubing will affect the thickness x₁ and x₂ of the neck brace.

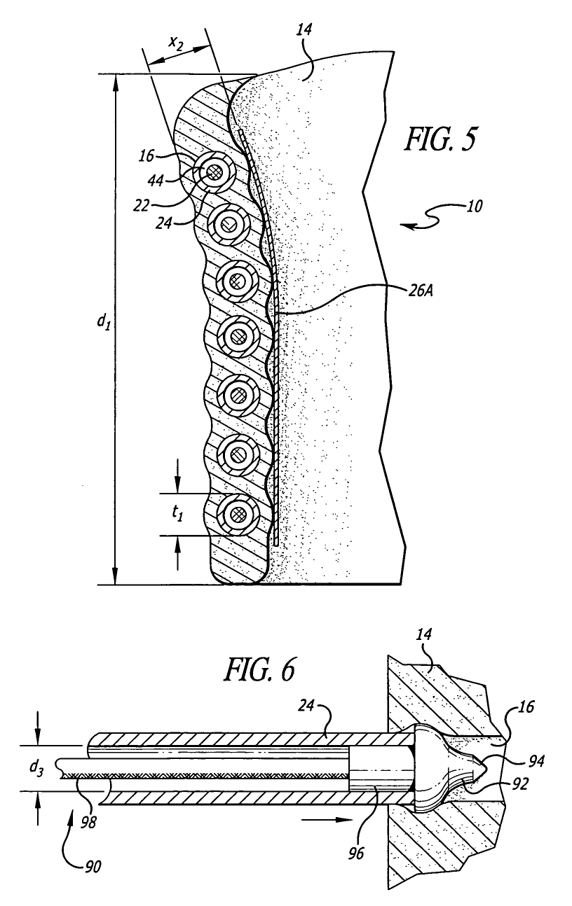

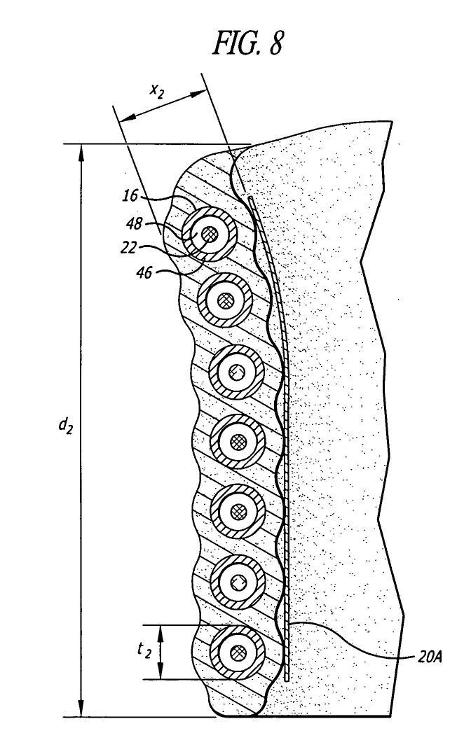

FIG. 6 is a partially exposed view showing tube threader 60 being used to insert a section of tubing 24 into a tunnel 16 of the soft and flexible neck brace 14. The tube threader 90 has an enlarged head 92 preferably with a pointed tip 94 and a sleeve region 96 behind the head and a push shaft 98 that has a outside diameter preferably smaller than the interior diameter d₃ of tubing 24. A section of tubing 24 can be placed on tube threader 90 behind its head 92 and used to position tubing within tunnel 16. Other tools can be used, and if desired, the head 62 and outside of tubing 24 can be wetted to ease sliding.

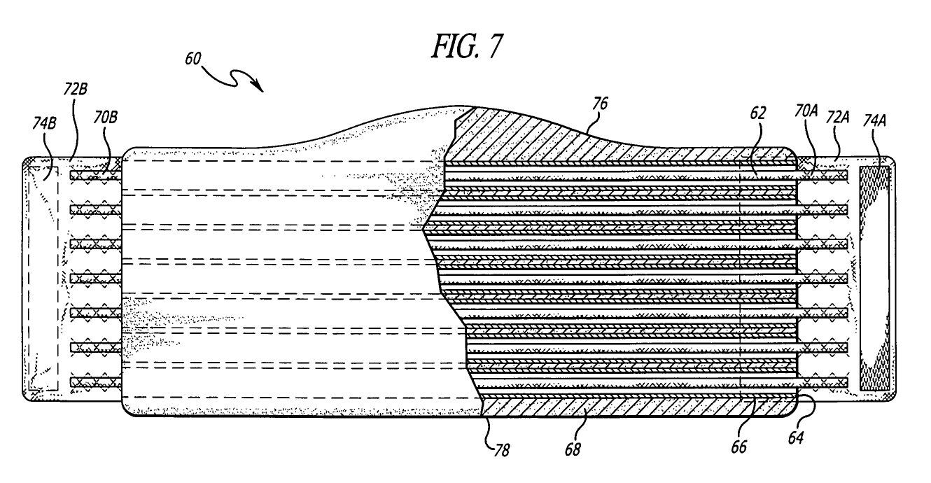

FIG. 7 is a partially exposed top plan view showing another embodiment of a soft and flexible neck brace 60 of the invention, laid out flat. This embodiment is similar to that shown in FIGS. 1-5, except that a plurality of lines 62 passing through-tubing 64 inserted into tunnels 66 in a soft and flexible portion 68. Ends 70A and 70B are affixed to engaging members 72A and 72B (e.g. by stitching, gluing, or other permanent physical attachment.) Engaging members 72A and 72B can comprise sections of fabric, and can have detachable attachment means 74A and 74B, respectively, located thereon, such as hook and loop material, snaps, hooks, and/or other means and materials, so that engaging members 72A and 72B can be held together, and thereby hold soft and flexible portion 68 around patient's neck. Although exemplary neck brace 60 is shown with all tubing 64 being the same diameter, it is possible to mix and match different diameter tubing to affect the distances between its top edge 76 and bottom edge 78. Also, it is also possible to provide stiff lining within tunnels 66 other than with tubing inserted therein.

Tubing 24, 48 and 64 can comprise bendable vinyl tubing, or other flexible tubing. Strong lines 22 and 72 such as parachute line functions well, although other strings, cords, threads and the like can be used. Alternately, the engaging members themselves can be affixed to the ends of the soft and flexible neck brace portion (e.g. by being partially imbedded therein, glued thereto, etc.) and therefore provide an alternate way to secure the neck brace around the neck of a wearer.

Although the invention has been shown and presented herein by means of certain embodiments of the display packages and shipping and display systems, it is to be understood that the invention is not limited thereto but may be variously embodied within the spirit and scope of the invention. Those of ordinary skill in the art will be able to identify various modifications which still remain within the ambit of the claims which follow.

1. A neck brace, comprising:
a brace body comprising flexible material with at least one tunnel formed therethrough having a first, smaller internal diameter when in an unstretched condition, and capable of being stretched to have a larger internal diameter; and
at least one elongate insert which can be received within the at least one tunnel, wherein the at least one elongate insert can have an outer diameter that can be smaller or larger than the first, smaller diameter of the at least one tunnel.

2. The neck brace of claim 1, wherein the brace body has two side edged and a top and bottom edge, and further comprising two engaging members that are affixable in the vicinity of the side edges for use in retaining the neck brace around a wearer's neck.

3. The neck brace of claim 2, wherein the two engaging members each comprise sections of material with complementary attachment means to permit the two sections of material to be connected together.

4. The neck brace of claim 2, wherein the two engaging means each comprise sections of material which are directed affixed to vicinity of the two sides of the body portion.

5. The neck brace of claim 1, wherein the at least one elongate insert comprises a flexible tube.

6. The neck brace of claim 2, wherein the at least one elongate insert comprises a flexible tube.

7. The neck brace of claim 6, further comprising strings that are passed through the tubes in the tunnels and connected with the engaging members.

8. The neck brace of claim 7, wherein the strings are sewn to the engaging members.

9. The neck brace of claim 7, wherein the engaging members have apertures formed therein, and the strings are threaded through the apertures to adjust of the spacing of the engaging members from the side edges of the brace body.

10. The neck brace of claim 1, wherein the brace body has a plurality of tunnels, two side edges, a bottom edge, and a top edge, and wherein a plurality of elongate inserts are provided that each comprise a flexible rods or tube, such that when flexible rods or tubes having a larger outer diameter than the first, smaller internal diameter of the tunnels in the brace body are inserted into the tunnels, the tunnels will be stretched, and the distance between the bottom edge and top edge of the brace body will be lengthened.

11. The neck brace of claim 1, wherein the at least one elongate insert comprises a flexible rod.

12. The neck brace of claim 1, further comprising a chin cup fixed on the brace body.

13. A neck brace, comprising:
a brace body comprising flexible material with two side edged and a top and bottom edge, and with a plurality of tunnels formed therethrough, each tunnel having a first, smaller internal diameter when in an unstretched condition, and each tunnel being capable of being stretched to have a larger internal diameter;
a plurality of elongate inserts which can be received within the plurality of tunnels, wherein the elongate inserts can have an outer diameter that can be smaller or larger than the first, smaller diameter of the plurality of tunnels; and
two engaging members that are affixable in the vicinity of the side edges for use in retaining the neck brace around a wearer's neck.

14. The neck brace of claim 13, wherein when inserts having a outer diameter larger than the first, smaller internal diameter of the tunnels in the brace body are inserted into the tunnels, the tunnels will be stretched, and the distance between the bottom edge and top edge of the brace body will be lengthened.

15. The neck brace of claim 13, wherein the two engaging members each comprise sections of material with complementary attachment means to permit the two sections of material to be connected together.

16. The neck brace of claim 13, wherein the two engaging means each comprise sections of material which are directed affixed to vicinity of the two sides of the body portion.

17. The neck brace of claim 13, wherein the plurality of elongate inserts comprises a flexible tube.

18. The neck brace of claim 17, further comprising strings that are passed through the tubes in the tunnels and connected with the engaging members.

19. The neck brace of claim 18, wherein the strings are sewn to the engaging members.

20. The neck brace of claim 18, wherein the engaging members have apertures formed therein, and the strings are threaded through the apertures to adjust of the spacing of the engaging members from the side edges of the brace body.

21. The neck brace of claim 13, wherein the at least one elongate insert comprises a flexible rod.

22. The neck brace of claim 13, further comprising a chin cup fixed on the brace body.

23. An adjustable neck brace system, comprising:
a brace body comprising flexible material with two side edged and a top and bottom edge, and with a plurality of tunnels formed therethrough, each tunnel having a first, smaller internal diameter when in an unstretched condition, and each tunnel being capable of being stretched to have a larger internal diameter;
a plurality of elongate inserts of various diameters which can be received within the plurality of tunnels, wherein the elongate inserts can have an outer diameter that can be smaller or larger than the first, smaller diameter of the plurality of tunnels; and
two engaging members that are affixable in the vicinity of the side edges for use in retaining the neck brace around a wearer's neck, wherein by selecting elongate inserts that have an outer diameter that is larger than the first, smaller internal diameter of the brace body in its unstretched condition, the tunnels will be stretched, and the distance between the bottom edge and top edge of the brace body will be lengthened.
